(19) United States
(12) Patent Application Publication
(10) Pub. No.: US 2014/0100470 A1
(43) Pub. Date: Apr. 10, 2014

(54) DIGITAL INSPIROMETER SYSTEM

(71) Applicant: Robert J. Perry, Oklahoma City, OK (US)

(72) Inventor: Robert J. Perry, Oklahoma City, OK (US)

(21) Appl. No.: 13/968,398

(22) Filed: Aug. 15, 2013

Related U.S. Application Data
(60) Provisional application No. 61/683,671, filed on Aug. 15, 2012.

(51) Int. Cl. A61B 5/087; A61B 5/091

(52) U.S. Cl. CPC A61B 5/087; A61B 5/091
USPC 600/538

(57) ABSTRACT

The present invention provides, in at least one embodiment, a system, device, and method for better instructions, feedback, and capture of inspirometer data. Inspirometers are typically not used correctly, so the device provides video, picture, and/or text instructions to users on how to use the inspirometer. Since many users do not inhale at the correct slow flow rate, the device has indicators telling the user whether his or her inhalation flow rate is too fast or too slow. Since many users do not use the inspirometer at all, the device captures electronic data including the total volume inhaled, flow rate data which indicates whether the user used the inspirometer correctly, and time stamps which indicate whether the users used the inspirometer regularly or at all. The device can also include spirometer features to capture exhaled breath data as well as inhaled breath data.

### CROSS REFERENCE TO RELATED APPLICATIONS

This application claims priority under 35 U.S.C. §119(e) to U.S. Provisional Application No. 61/683,671, filed Aug. 15, 2012, and entitled “Digital Inspirometer System,” the disclosure of which is hereby incorporated by reference in its entirety.

### BACKGROUND OF THE INVENTION

1. Field of Invention

The invention relates generally to inspirometers, and more particularly, to a digital inspirometer system with improved instructions, feedback, and captured electronic data.

2. Description of Related Art

Inspirometers measure the volume of air that a person inhales in with a deep breath. Inspirometers are used in diagnostic measures to determine the degree of lung compromise or airway obstructions, as in asthma or chronic obstructive pulmonary disease.

Inspirometers are also given to surgical patients during their post-operative recovery period as incentive to the user to take slow deep breaths. Breathing in deeply and expanding the lungs fully after surgery is necessary for clearing the lungs of secretions from anesthesia use and to prevent pneumonia. Post-operative patients are often reluctant to breathe in deeply and aerate their lung bases properly when they have painful surgical wounds that cause them to naturally use shallow breathing techniques.

One type of inspirometer is an incentive spirometer, which is a medical device used to help users improve the functioning of their lungs. It is provided to patients who have had any surgery that might jeopardize respiratory function, particularly surgery to the lungs themselves, but also commonly to patients recovering from cardiac or other surgery involving extended time under anesthesia and prolonged in-bed recovery. The incentive spirometer is also issued to patients recovering from rib damage to help minimize the chance of fluid build-up in the lungs. It can be used as well by wind instrument players, who want to improve their air flow.

To use an inspirometer, the user breathes in from the inlet opening as slowly and as deeply as possible. Then, the user holds his or her breath for two to six seconds. This provides back pressure which pops open the user's alveoli, which are tiny air filled sacs arranged in clusters in the user's lungs. The inhalation is similar to a yawn. An indicator provides a gauge of how well the user's lung or lungs are functioning, by indicating sustained inhalation vacuum. The user is generally asked to do many repetitions a day while measuring his or her progress by way of the gauge.

There are spirometer technologies, but these are different than inspirometers technologies. A spirometer refers to measuring measures air exhaled, as opposed to inhaled, which is measured by an inspirometer. These spirometers measure the volume of air expired by the lungs and the corresponding respiration flow rates over a specified period. To measure the flow rates, the spirometer often uses a precision differential pressure transducer.

Spirometer technologies include whole body plethysmograph, pneumotachometers, fully electronic spirometers, peak flow meters, windmill-type spirometers, and tilt-compensated spirometers. Electronic spirometers have been developed that compute airflow rates in a channel without the need for fine meshes or moving parts. They operate by measuring the speed of the airflow with techniques such as ultrasonic transducers, or by measuring pressure difference in the channel. These spirometers have greater accuracy by eliminating the momentum and resistance errors associated with moving parts such as windmills or flow valves for flow measurement. They also allow improved hygiene between users by allowing fully disposable air flow channels.

U.S. Pat. No. 5,518,002 to Wolfe is a peak flow spirometer. This portable electronic spirometer device is hand held and analyzes the strength of the exhalation of a user under a doctor's care. The electronic spirometer device is designed to sense and measure exhaled air flow rate and exhaled breath temperature, determine air flow volume, and record and display the respiratory movement of the user for helping make medication recommendations. The device is also designed to give interpretive feedback and recommendations which are pre-programmed by the doctor for the user and to detect beforehand a possible chronic episode, for example a pending asthmatic attack, and alert the user to take necessary medication to avert the episode. The portable device may be periodically connected to a computer system to up-load stored information and provide a chronological report stored therein for analysis by the doctor. The stored data can be transferred from the spirometer device via telephone and modem to the doctor's office.

Wolfe's spirometer is often referred to as a peak flow meter. The peak flow meter is used to diagnose and monitor air trapping diseases such as asthma, emphysema, bronchitis, etc. These diseases are called air trapping diseases because the air can get in, but has trouble getting out. Wolf's peak flow meter is designed to measure the peak flow and volume that a user can “exhale” and based on these measurements a doctor can tell the degree of trapping. The user's breathes as “fast” as they can for a first few seconds.

However, Wolfe is not an inspirometer. Wolfe measures expiration, which is breathing air out from the lungs, as opposed to inspiration, which is the inhalation of air into the lungs. A peak flow meter requires the user to exhale and breathe very fast, which is the opposite requirements for an inspirometer, which require the user to inhale very slow to expand the lungs. Also, Wolfe's recorded information is not used to provide immediate feedback to the user regarding their technique, or even provide the data to the user at all.

There are several problems with inspirometers. Research has indicated that 90% of users do not use them correctly. For example, many users inhale too fast. The users may inhale too fast because they do not know the correct flow rate or because they were not instructed properly. The users may inhale too fast in an attempt to cheat the inspirometer and get a higher total volume reading. Other users assume the inspirometer is disposable and throw it away after limited use. Other users do not use the inspirometer at all.

### SUMMARY OF THE INVENTION

The present invention provides, in at least one embodiment, a system, device, and method for better instructions, feedback, and capture of inspirometer data. Inspirometers are typically not used correctly, so the device provides video, picture, and/or text instructions to users on how to use the inspirometer. Since many users do not inhale at the correct slow flow rate, the device has indicators telling the user whether his or her inhalation flow rate is too fast or too slow. Since many users do not use the inspirometer at all, the device captures electronic data including the total volume inhaled, flow rate data which indicates whether the user used the inspirometer correctly, and time stamps which indicate whether the users used the inspirometer regularly or at all. The device can also include spirometer features to capture exhaled breath data as well as inhaled breath data.

In one embodiment, a system comprises: a digital inspirometer comprising an inlet configured to provide air inhaled by a patient, and a sensor configured to capture a plurality of measured variables from the air inhaled air; and an electronic display configured to display instructions to the patient, configured to display the plurality of measured variables, and configured store the plurality of measured variables in a database. The plurality of measured variables may comprise a time, a total volume, and a flow rate. The database may be coupled to the electronic display. The sensor may comprise a bidirectional fan. The digital inspirometer may further comprise a handle.

In another embodiment, a method comprises the steps of: providing instructions to a patient; providing air inhaled by a patient; capturing a plurality of measured variables from the air inhaled air; displaying the plurality of measured variables; and storing the plurality of measured variables in a database.

In a further embodiment, a device comprises: an input configured to receive captured electronic data, wherein the captured electronic data comprises a flow rate of inhaled air; a display coupled to the input, wherein the display shows the captured electronic data or instructions to a user; an indicator coupled to the input, wherein the indicator provides feedback to the user regarding the flow rate; and a database coupled to the input, wherein the database stores the captured electronic data.

An advantage of the present invention is that the device better instructs the user how to use the inspirometer. By the device having an electronic display, the user can be shown pictures, videos, and/or text of the proper technique.

Another advantage of the invention is that it provides immediate feedback to the user. The user can visually see indicators during use, whether the air is being inhaled or exhaled too slow or too fast. A further advantage of the invention is the electronic capturing of electronic data. By collecting the data electronically, the data can more easily be seen by the user, stored, and provided to a medical professional. Having real time feedback allows the medical professional to monitor lung function accurately enough to be able to predict pneumonia development 24-48 hour before changes can be seen on an x-ray.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, and the advantages thereof, reference is now made to the ensuing descriptions taken in connection with the accompanying drawings briefly described as follows:

FIG. 1 illustrates a digital inspirometer system according to an embodiment of the invention;

FIG. 2 illustrates a device of the system according to an embodiment of the invention;

FIG. 3 illustrates an inspirometer of the system according to an embodiment of the invention; and

FIG. 4 illustrates the process of instructing a user and capturing electronic data according to an embodiment of the invention.

### DETAILED DESCRIPTION OF EMBODIMENTS

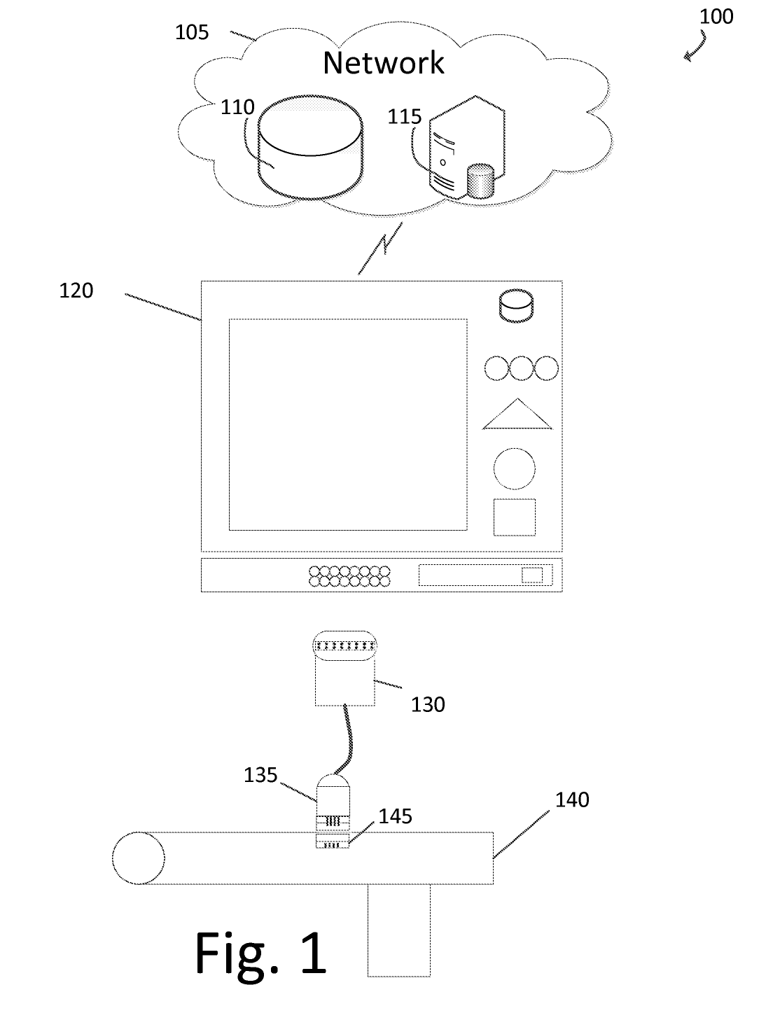

Further features and advantages of the invention, as well as the structure and operation of various embodiments of the invention, are described in detail below with reference to the accompanying FIGS. 1-4, wherein like reference numerals refer to like elements. Although the device is illustrated as a monitor-like configuration, the device can include other forms (e.g., an App on a mobile phone). Although this invention has novel aspects with the inspirometer features alone, a dual inspirometer and spirometer embodiment adds further value.

The present invention provides, in at least one embodiment, a system, device, and method which provides an electronic display for better instructions to users on how to use the device, indicators which provide immediate feedback on whether the user is using the device correctly, and electronic connections and circuitry giving the device the ability to receive and send the captured data electronically. The captured data indicates whether the user is using the device correctly (e.g., slow flow rate) or at all (e.g., through time stamps).

FIG. 1 illustrates a digital inspirometer system 100 according to an embodiment of the invention. The system 100 comprises a network 105 having a database 110 and a server 115, a device 120, connectors 130 and 135, and an inspirometer 140 having an electronic output 145. The system 100 provides user instructions, provides immediate user feedback, and captures data electronically.

The network 105 (e.g., the Internet) stores and communicates the data captured by the device 120. The network 105 can include one or more databases 110 to collect and organize the data in digital form and the servers 115 to respond to requests across the network 105. The network 105 can be a collection of computers and other hardware components interconnected by communications channels that allow sharing of resources and information.

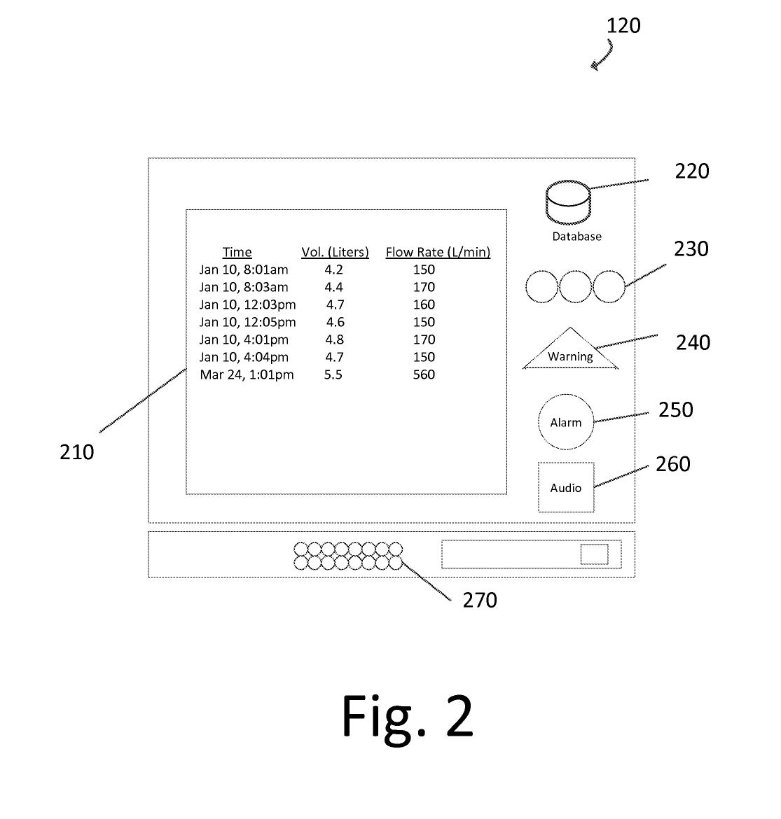

The device 120 conveys visual information to the user including instructions, immediate feedback on technique and results, and captures electronic data. For example, the variables may include volume, flow rate, date and time of actual use, alarm time for when the user should use the inspirometer, temperature of air, alcohol content, acetone (which is an indicator of Betaketoacidosis which is often associated with Diabetics not taking their insulin), ketones on breath (meaning the patient is not taking insulin), carbon dioxide (CO2), etc. The device 120 is discussed further with respect to FIG. 2.

The connectors 130 and 135 provide an electronic connection between the device 120 and the inspirometer 140. The connectors 130 and 135 are electro-mechanical devices to join electrical circuits. The connection is intended to be temporary and detachably coupled for portable equipment, but could also be permanent such as electrical wiring.

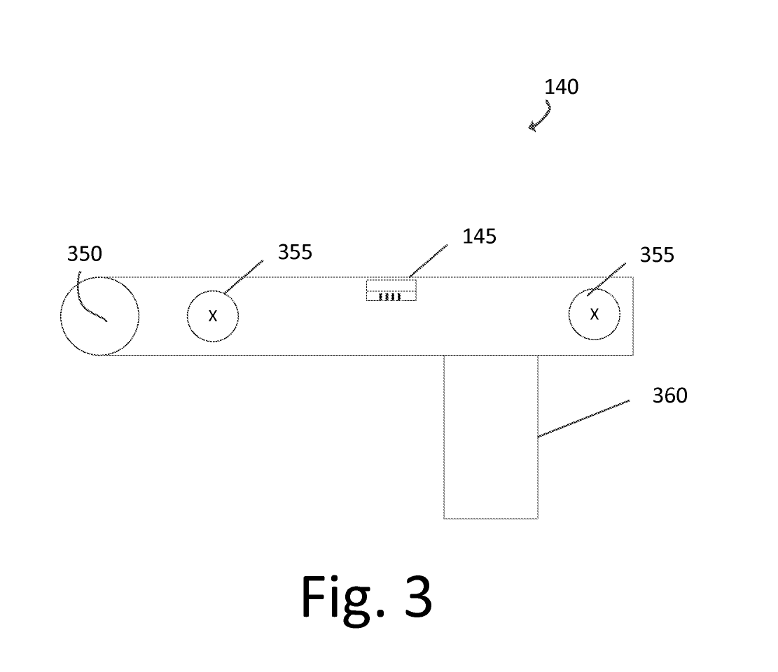

The inspirometer 140 (e.g., digital inspirometer, dual inspirometer/spirometer, etc.) measures inspiration variables and outputs them to the electronic device 120. The inspirometer 140 can also include hardware and software such that it also captures spirometer data. The inspirometer 140 is discussed further with respect to FIG. 3. The electronic output 145 is configured to detachably couple to the connector 135 and send data from the inspirometer 140 to the device 120.

FIG. 2 illustrates the device 120 of the system 100 according to an embodiment of the invention. The device 120 comprises a display 210, a database 220, one or more indicators 230, a warning indicator 240, an alarm indicator 250, audio 260, and input/output connectors 270.

The device 120 provides better instructions and immediate capturing of electronic data. The device 120 conveys inspirometer instructions much better than conventional methods. The device 120 comprises hardware and software. The hardware can comprise a processor and memory, and a graphics card.

In one embodiment, the device 120 has a form factor similar to an MP-3 player. In another embodiment, the device 120 is a privacy secured mobile phone application (e.g., iPhone App, BlackBerry App). Currently, only BlackBerry applications are Health Insurance Portability and Accountability Act (HIPAA) qualified. It is important that private medical data is communicated securely.

The display 210 (e.g., screen, electronic display, etc.) is configured to display instructions and captured electronic data. The instructions can be pictures or videos and can be in color. The pictures can include flow charts, sample users pictures, etc. The videos can be animations, cartoons, instructional videos, etc. The device 120 addresses a key problem with inspirometers, which is a misunderstanding of how to use them.

The display 210 illustrates the following captured data: time, volume, and flow rate. In addition, the display 210 may capture the number of breaths, the amount of time the breath is held after inspiration and before exhalation (also known as the pause). By capturing this data, a medical professional can see if the user is using the inspirometer at all or regularly and can check if the volume data and the flow rate data are acceptable. In a conventional inspirometer, this data is not immediately available to the user or the medical professional. As illustrated, the user may go several months without using the inspirometer 140, and as illustrated in the last measurement, may inhale way too fast.

The database 220 provides memory storage on the device 120. The device 120 can store captured data in the database 220 until it is provided to a medical professional. The database 220 also has instructional videos built in. The instructions can be cartoons designed for kids, textual instructions designed for adults, etc.

The indicators 230 visually indicate to the user whether the flow rate is too fast, too slow, or correct. For example, light emitting diodes (LEDs) can light up corresponding to the flow rate, where an acceptable flow rate is shown in green, and an unacceptable flow rate is shown in red. In another embodiment, the indicators 230 correspond with the flow rate, with text above the indicators 230 showing whether the flow rate is correct. In addition to flow rate, the indicators 230 can pick up on the number of breathes done correctly, the number which are too weak, and the number which are too fast, and the ratio of the number of breathes attempted to the number of breathes completed correctly.

The indicators 230 provide immediate feedback on whether the inspirometer 140 is being used correctly. Often the user will inhale too quickly, when they should inhale slow and steady. Slow controlled inspirations to get the lungs to fill with air to break up the fluid that gets trapped in the distal areas of the lungs which is where an infection may initially grow.

The warning indicator 240 informs the user that the flow rate is not correct. The alarm indicator 250 informs the user that it is time to use the inspirometer 140. In the illustrated embodiment, the alarm is set multiple times a day.

The audio 260 provides sound for the indicators, alarm, and/or instructions. The audio, along with the display 210, help better convey instructions and provide immediate feedback to the user. The input/output connectors 270 (e.g., input, output, etc.) are capable of connecting to the inspirometer 140, a computer, a mobile device, peripheral electronics, and wirelessly to the network 105.

FIG. 3 illustrates the inspirometer 140 of the system 100 according to an embodiment of the invention. The inspirometer 140 comprises the electronic output 145, an inlet 350, sensors 355, and a handle 360. The inspirometer 140 can be just an inspirometer. However, in a preferred embodiment, the inspirometer 140 contains the dual features of both an inspirometer and spirometer.

The inspirometer 140 records if the user is using the device 120 for inspiratory breathing, and how well they are using the device 120 during the inspiratory breathing. Conversely, the spirometer measures the exhalation. This exhalation function can be added in software and/or hardware. The user could press one button for the inspirometer function, which may be the primary function of the inspirometer 140, and another button to treat the inspirometer 140 like a peak flow meter spirometer.

The inlet 350 is configured to receive air exhaled from a user or transfer air inhaled by the user. The inlet 350 can be a mouthpiece, a tube, or other apparatus, the design of which is known by those with skill in the art.

The sensors 355 can be unidirectional sensors for the embodiment where the inspirometer 140 is just an inspirometer. However, for the embodiment where the inspirometer 140 is a dual inspirometer and spirometer, the sensors 355 are bidirectional (e.g., a bidirectional fan, a thermistor, etc.). The sensors 355 measure variables related to the user's inhalation or exhalation.

The handle 360 provides the user with a grip to hold the inspirometer 140. The handle can have grips or be shaped such that the user can easily grab the handle and hold. For example, the handle 360 can be shape like the handle of a gun.

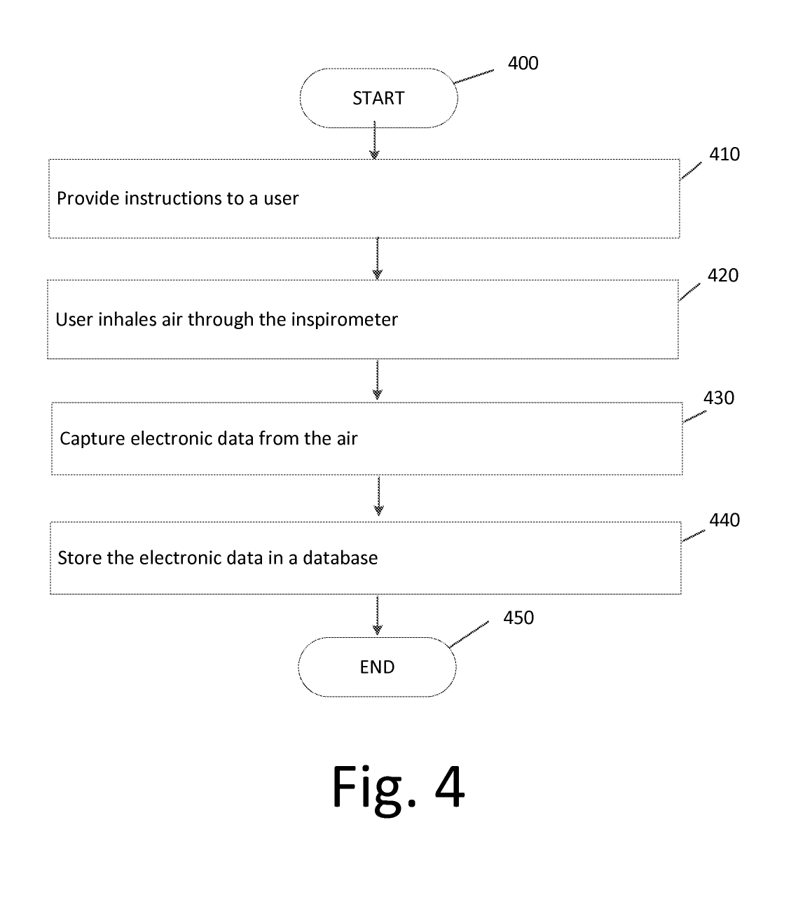

FIG. 4 illustrates the process of instructing a user and capturing electronic data according to an embodiment of the invention. The process starts at step 400. At step 410, the device 120 provides instructions to a user. These instructions can be conveyed better than conventional methods through videos, pictures, and/or text. At step 420, the user inhales air through the inspirometer 140. In the dual inspirometer and spirometer embodiment, the user can inhale or exhale air. The inspirometer 140 captures electronic data from the air at step 430. This electronic data can include, for example, volume, flow rate, date, time, an alarm time indicating a time to use, etc. At step 440, the device 120 stores the electronic data in the database 220 and/or on the network 105. The process may be repeated recursively a number of times and ends at step 450.

It is to be recognized that depending on the embodiment, certain acts or events of any of the methods described herein can be performed in a different sequence, may be added, merged, or left out altogether (for example, not all described acts or events are necessary for the practice of the method). Moreover, in certain embodiments, acts or events may be performed concurrently, for example, through multi-threaded processing, interrupt processing, or multiple processors, rather than sequentially.

The inspirometer 140 can perform many tests including lung function tests. Lung function tests (also called a pulmonary function tests, or PFTs) check how well the user's lungs work. The tests determine how much air the user's lungs can hold, how quickly the user can move air in and out of the user's lungs, and how well the user's lungs put oxygen into and remove carbon dioxide from the user's blood. The tests can diagnose lung diseases, measure the severity of lung problems, and check to see how well treatment for a lung disease is working.

Other tests to determine lung function include tests on residual volume, gas diffusion tests, body plethysmography, inhalation challenge tests, and exercise stress tests. Spirometry is the first and most commonly done lung function test. It measures how much and how quickly you can move air out of your lungs. For this test, the user breathes into a mouthpiece attached to a recording device (spirometer). The information is collected by the spirometer may be printed out on a chart called a spirogram.

The inspirometer 140 can measure many different type of parameters, including forced vital capacity, forced expiratory volume, forced expiratory flow 25% to 75%, peak expiratory flow (PEF), maximum voluntary ventilation, slow vital capacity, total lung capacity, functional residual capacity, residual volume, and expiratory reserve volume.

Forced vital capacity (FVC) measures the amount of air a user can exhale with force after the user inhales as deeply as possible. Forced expiratory volume (FEV) measures the amount of air the user can exhale with force in one breath. The amount of air the user exhales may be measured at 1 second (FEV1), 2 seconds (FEV2), or 3 seconds (FEV3). FEV1 divided by FVC can also be determined. Forced expiratory flow 25% to 75% measures the air flow halfway through an exhale. Peak expiratory flow (PEF) measures how quickly the user can exhale. It is usually measured at the same time as the user's forced vital capacity (FVC). Maximum voluntary ventilation (MVV) measures the greatest amount of air the user can breathe in and out during one minute. Slow vital capacity (SVC) measures the amount of air the user can slowly exhale after the user inhales as deeply as possible.

Total lung capacity (TLC) measures the amount of air in your lungs after the user inhales as deeply as possible. Functional residual capacity (FRC) measures the amount of air in the user's lungs at the end of a normal exhaled breath. Residual volume (RV) measures the amount of air in the user's lungs after the user has exhaled completely. It can be done by breathing in helium or nitrogen gas and seeing how much is exhaled. Expiratory reserve volume (ERV) measures the difference between the amount of air in your lungs after a normal exhale (FRC) and the amount after you exhale with force (RV).

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

What is claimed is:

1. A system comprising:
a digital inspirometer comprising an inlet configured to provide air inhaled by a patient, and a sensor configured to capture a plurality of measured variables from the air inhaled air; and
an electronic display configured to display instructions to the patient, configured to display the plurality of measured variables, and configured store the plurality of measured variables in a database.

2. The system of claim 1, wherein the plurality of measured variables comprises a time, a total volume, and a flow rate.

3. The system of claim 1, wherein the database is coupled to the electronic display.

4. The system of claim 1, wherein the sensor comprises a bidirectional fan.

5. The system of claim 1, wherein the digital inspirometer further comprises a handle.

6. A method comprising:
providing instructions to a patient;
providing air inhaled by a patient;
capturing a plurality of measured variables from the air inhaled air;
displaying the plurality of measured variables; and
storing the plurality of measured variables in a database.

7. A device comprising:
an input configured to receive captured electronic data, wherein the captured electronic data comprises a flow rate of inhaled air;
a display coupled to the input, wherein the display shows the captured electronic data or instructions to a user;
an indicator coupled to the input, wherein the indicator provides feedback to the user regarding the flow rate; and
a database coupled to the input, wherein the database stores the captured electronic data.
